(19) United States
(12) Patent Application Publication
(10) Pub. No.: US 2026/0221266 A1
(43) Pub. Date: Jul. 30, 2026

(54) IMAGE PROCESSING APPARATUS AND PHASE CORRECTION METHOD

(71) Applicant: CANON KABUSHIKI KAISHA, Tokyo (JP)

(72) Inventors: Shohei HAMANAGA, Fukuoka Fukuoka (JP); Hideaki KUTSUNA, Kawasaki Kanagawa (JP); Mitsuhiro BEKKU, Utsunomiya Tochigi (JP); Masahiro ABE, Yokohama Kanagawa (JP); Kensuke SHINODA, Otawara Tochigi (JP)

(73) Assignee: CANON KABUSHIKI KAISHA, Tokyo (JP)

(21) Appl. No.: 19/449,609

(22) Filed: Jan. 15, 2026

(30) Foreign Application Priority Data
Jan. 28, 2025 (JP) 2025-012459

(51) Int. Cl. G16H 30/40; G06T 3/40; G06T 5/70; G16H 30/20

(52) U.S. Cl. CPC G16H 30/40; G06T 3/40; G06T 5/70; G16H 30/20

(57) ABSTRACT

An image processing apparatus according to an embodiment includes a processing circuitry. The processing circuitry configured to: perform processing for reducing a background phase included in complex data, the complex data being at least one piece of complex data among multiple pieces of complex data obtained by scanning a subject by a magnetic resonance imaging apparatus; calculate complex average data by performing averaging processing on the multiple pieces of complex data including the complex data in which the background phase is reduced; and perform post-processing on the complex average data.

### CROSS-REFERENCE TO RELATED APPLICATIONS

This application is based upon and claims the benefit of priority from Japanese Patent Application No. 2025-012459, filed Jan. 28, 2025, the entire contents of which are incorporated herein by reference.

### FIELD

Embodiments described herein relate generally to an image processing apparatus and a phase correction method.

### BACKGROUND

A magnetic resonance imaging (MRI) apparatus is an imaging apparatuses that excites nuclear spins of a subject placed in a static magnetic field using radio frequency (RF) signals at the Larmor frequency and reconstructs magnetic resonance (MR) data based on MR signals generated from the subject in response to the excitation, to thereby generate an MR image. The MR data is complex data including a real part and an imaginary part.

In some cases, the number of excitations (Nex) for MR data may be increased to improve a signal-to-noise (S/N) ratio in an MR image. In the related art, a magnitude image obtained by performing averaging processing on MR data corresponding to the Nex based on absolute value (magnitude) data indicating the square root of the sum of squares of a real part and an imaginary part is generated as an MR image. On the other hand, it is known that averaging processing based on complex data, as compared with the averaging processing based on absolute value data, reduces background noise in the MR image, which leads to an improvement in the S/N ratio.

In a diffusion weighted imaging (DWI) method, for example, a background phase in a phase image based on data representing a phase angle of the real part and the imaginary part may vary for each accumulation number (e.g., shot) within the Nex due to an influence of application of a motion probing gradient (MPG).

In a case where the background phase varies for each accumulation number in this manner, execution of the averaging processing based on complex data may cause degradation in image quality of an MR image to be generated, or may cause an unexpected decrease in the S/N ratio.

### BRIEF DESCRIPTION OF THE DRAWINGS

FIG. 1 is a block diagram illustrating an example of an overall configuration of an image processing apparatus according to an embodiment;

FIG. 2 is an explanatory diagram illustrating generation of a magnetic resonance (MR) image based on multiple pieces of MR data according to a comparative example;

FIG. 3 is a flowchart illustrating an example of operation performed by the image processing apparatus according to the embodiment;

FIG. 4 is an explanatory diagram illustrating processing with regard to a background phase according to the embodiment;

FIG. 5 is an explanatory diagram illustrating generation of an MR image based on multiple pieces of MR data according to the embodiment;

FIG. 6 is another explanatory diagram illustrating generation of an MR image based on multiple pieces of MR data according to the embodiment;

FIG. 7 is a flowchart illustrating an example of operation in a case where resolution enhancement processing is executed in post-processing illustrated in FIG. 6;

FIGS. 8A, 8B, 8C, 8D, 8E, 8F, 8G, and 8H each illustrate an example of a change in image quality of an MR image according to the type of averaging processing and the number of excitations (Nex);

FIGS. 9A and 9B are graphs each illustrating an example of a change in image quality of an MR image according to the type of averaging processing and the Nex;

FIG. 10 is a block diagram illustrating an example of an overall configuration of an image processing apparatus according to a modified example of the embodiment;

FIG. 11 is a flowchart illustrating an example of operation performed by the image processing apparatus according to the modified example of the embodiment;

FIGS. 12A and 12B are explanatory diagrams each illustrating a discontinuous phase change; and

FIG. 13 is a block diagram illustrating an example of an overall configuration of a magnetic resonance imaging (MRI) apparatus according to the embodiment.

### DETAILED DESCRIPTION

An image processing apparatus according to an embodiment includes a processing circuitry. The processing circuitry configured to: perform processing for reducing a background phase included in complex data, the complex data being at least one piece of complex data among multiple pieces of complex data obtained by scanning a subject by a magnetic resonance imaging apparatus; calculate complex average data by performing averaging processing on the multiple pieces of complex data including the complex data in which the background phase is reduced; and perform post-processing on the complex average data.

Various Embodiments will be described hereinafter with reference to the accompanying drawings.

In the drawings, the identical elements are denoted by the same reference numerals, and repeated descriptions thereof are omitted.

### (Image Processing Apparatus According to Embodiment)

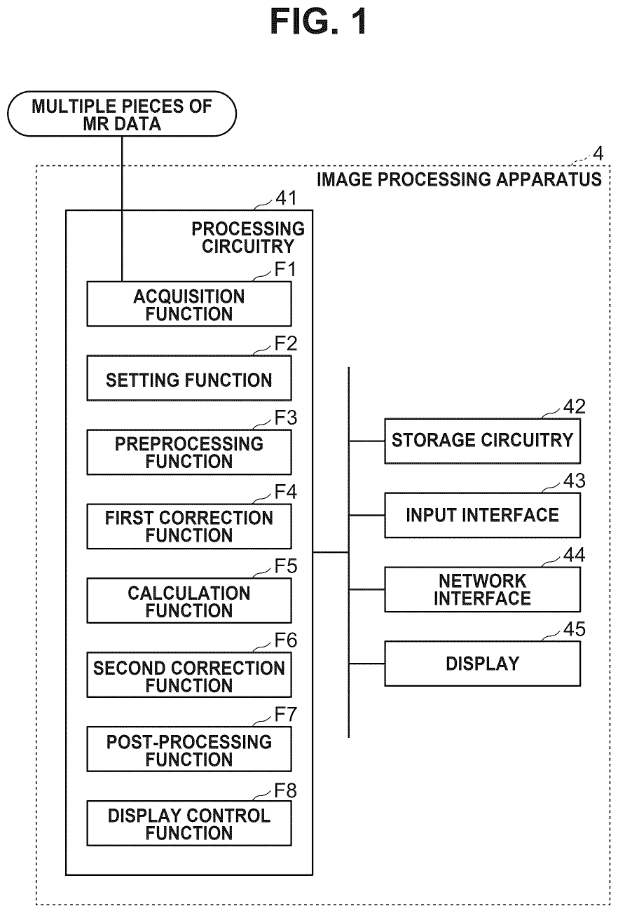

FIG. 1 is a block diagram illustrating an example of an overall configuration of an image processing apparatus 4 according to an embodiment. The image processing apparatus 4 performs processing related to a background phase of a magnetic resonance (MR) image. The image processing apparatus 4 includes processing circuitry 41, storage circuitry 42, an input interface 43, a network interface 44, and a display 45.

The display 45 is, for example, a display device such as a liquid crystal display, an organic light emitting diode (OLED) display panel, a plasma display panel, or an organic electroluminescence (EL) panel. The display 45 displays various kinds of information and images under the control of the processing circuitry 41. The display 45 may serve not only as a display device, but also as a graphical user interface (GUI) that functions as an input device.

The network interface 44 implements various kinds of information communication protocols depending on the form of a network. The network interface 44 controls communication according to various protocols and performs wired or wireless communication with various apparatuses connected to the network to exchange various kinds of information and data.

The input interface 43 includes an input device configured to be operated by a user, and input circuitry configured to process signals from the input device. Examples of the input device include a trackball, a switch, a mouse, a keyboard, a touch-pad including an operating surface on which a user touches to perform an input operation, a touch screen in which a display screen and a touch-pad are integrated, a non-contact input device using an optical sensor, and an audio input device. When the input device is operated by the user, the input circuitry generates a signal corresponding to the operation and outputs the signal to the processing circuitry 41.

The storage circuitry 42 is a storage medium readable by a processor, such as a semiconductor memory element (e.g., a random access memory (RAM), a flash memory), a hard disk, or an optical disk device. The storage circuitry 42 may be a portable medium such as a universal serial bus (USB) memory or a digital versatile disk (DVD). The storage circuitry 42 stores various kinds of information and data. The storage circuitry 42 stores various programs to be executed by a processor of the processing circuitry 41 and data required to execute programs.

The processing circuitry 41 controls operations of the image processing apparatus 4 in an integrated manner. The processing circuitry 41 is a processor that reads out various processing programs that are stored in the storage circuitry 42 or directly incorporated into the processing circuitry 41, and executes the processing programs to thereby implement various functions. The processor that is the processing circuitry 41 executes various programs to thereby execute an acquisition function F1, a setting function F2, a preprocessing function F3, a first correction function F4, a calculation function F5, a second correction function F6, a post-processing function F7, and a display control function F8 corresponding to the various programs.

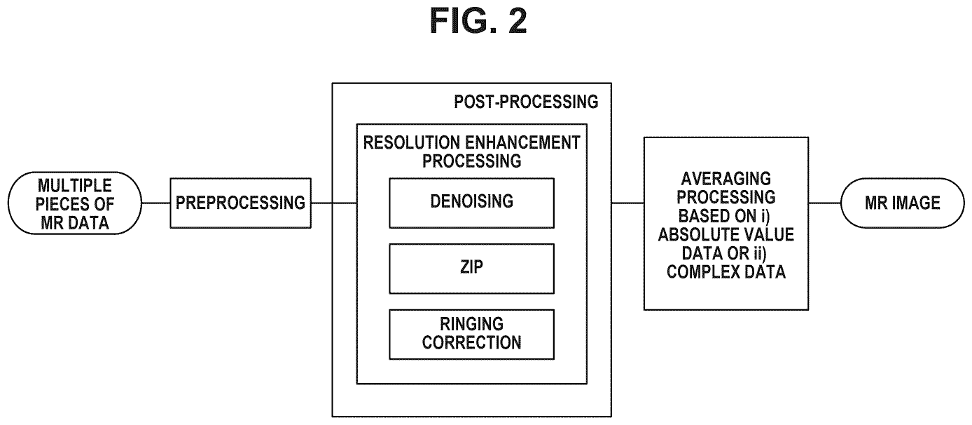

A comparative example illustrated in FIG. 2 is an example of operation with regard to generation of an MR image based on multiple pieces of MR data according to the related art. Differences between the comparative example and the embodiment will be described with reference to FIG. 2.

In the related art, after post-processing for improving image quality has been performed on each of the multiple pieces of MR data corresponding to the number of excitations (Nex), averaging processing is performed to generate an MR image. However, when various types of post-processing are performed on the multiple pieces of MR data prior to the averaging processing, there is a risk that errors caused by the post-processing will accumulate and image quality degradation such as image blurring will occur, as compared to a case where the post-processing is performed on one piece of MR data in which a signal-to-noise (S/N) ratio has been improved by the averaging processing.

Further, in the related art, since image processing is performed on the MR data corresponding to the Nex prior to the averaging processing, there is a risk that a processing speed until generation of an MR image becomes slow. Specifically, the processing time for generating an MR image becomes longer when the post-processing is performed on the multiple pieces of MR data obtained prior to the averaging processing than when the post-processing is performed on one piece of MR data obtained after the averaging processing. Therefore, the image processing apparatus 4 according the embodiment performs averaging processing at a timing prior to post-processing, thereby reducing the processing time until generation of an MR image.

The averaging processing can be performed based on absolute value data or complex data. For example, in a diffusion weighted imaging (DWI) method, a background phase can significantly vary for each accumulation number (e.g., shot) due to an influence of application of a motion probing gradient (MPG). For example, differences in background phase can be caused by static magnetic field inhomogeneity, an eddy current, or a motion of a patient, and the background phase can change even within scans of the same subject.

In a case where the background phase varies for each accumulation number, when the averaging processing based on complex data is performed, degradation in image quality of an MR image to be generated can be caused, or an unexpected decrease in the S/N ratio can be caused. For example, if the averaging processing is performed on pieces of MR data with opposite phases from each other, signals are cancelled out. Accordingly, in the related art, the averaging processing on the multiple pieces of MR data corresponding to the Nex is performed based on the absolute value data in many cases.

On the other hand, it is known that in the averaging processing based on complex data, background noise in an MR image is reduced compared to that in the averaging processing based on absolute value data, which leads to an improvement in the S/N ratio. Further, in the case where the background phase varies for each accumulation number, when the averaging processing based on complex data is performed after the background phase is reduced, it possible to reduce an unexpected decrease in the S/N ratio.

However, the consistency between amplitude (or magnitude) and phase in complex average data after the averaging processing is lost due to a reduction of the background phase. In a case where phase information is required in subsequent post-processing, the post-processing is performed on complex average data in a state where the consistency between the amplitude and the phase is lost, which may lead to degradation in quality of an MR image to be generated.

For example, when Zero Filling Interpolation (ZIP) processing is performed as the post-processing on the complex average data in a state where the consistency between the amplitude and the phase is lost due to the reduction of the background phase, an artifact such as a ringing artifact may appear in the MR image. For this reason, the image processing apparatus 4 according to the embodiment adds a pseudo-phase after the averaging processing to maintain the consistency between the amplitude and the phase of the complex average data after the averaging processing in the post-processing.

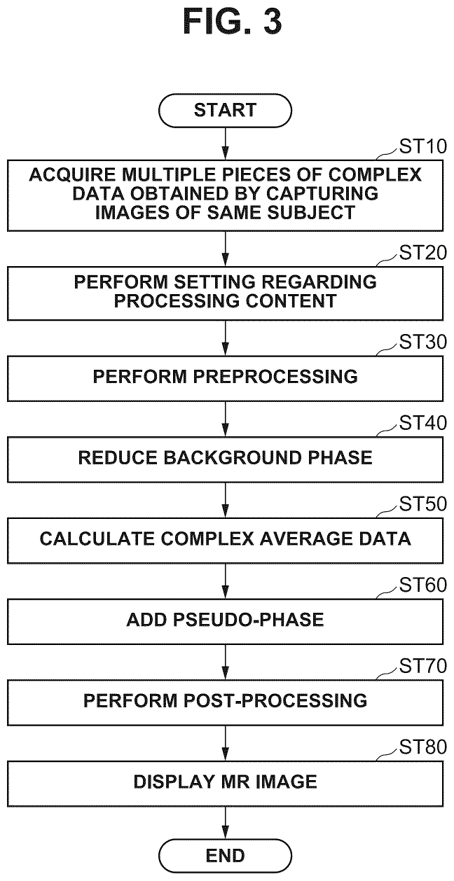

An example of operation with regard to generation of an MR image based on multiple pieces of MR data by the image processing apparatus 4 according to the embodiment will be described with reference to a flowchart illustrated in FIG. 3 and FIGS. 4 to 7.

In step ST10, the acquisition function F1 acquires multiple pieces of complex data obtained by capturing images of the same subject.

In step ST20, the setting function F2 performs setting of whether to execute at least one of processing related to a background phase and resolution enhancement processing. The processing related to the background phase includes processing for reducing the background phase and processing for adding the pseudo-phase. The processing related to the background phase and the resolution enhancement processing will be described in detail below.

The setting function F2 can perform the setting of whether to execute at least one of the processing related to the background phase and the resolution enhancement processing prior to the processing related to the background phase. For example, a selection screen for ON or OFF of settings may be displayed on the display 45, and the setting function F2 may perform the setting of whether to execute at least one of the processing related to the background phase and the resolution enhancement processing according to an instruction from the user who has performed an input operation via the input interface 43. The setting function F2 may determine and perform the setting of whether to execute at least one of the processing related to the background phase and the resolution enhancement processing based on an imaging condition. The setting function F2 may perform the setting of whether to execute at least one of the processing related to the background phase and the resolution enhancement processing after the processing related to the background phase.

In step ST30, the preprocessing function F3 performs preprocessing on the multiple pieces of complex data. The preprocessing function F3 may perform, for example, k-space filtering processing and parallel imaging processing as the preprocessing.

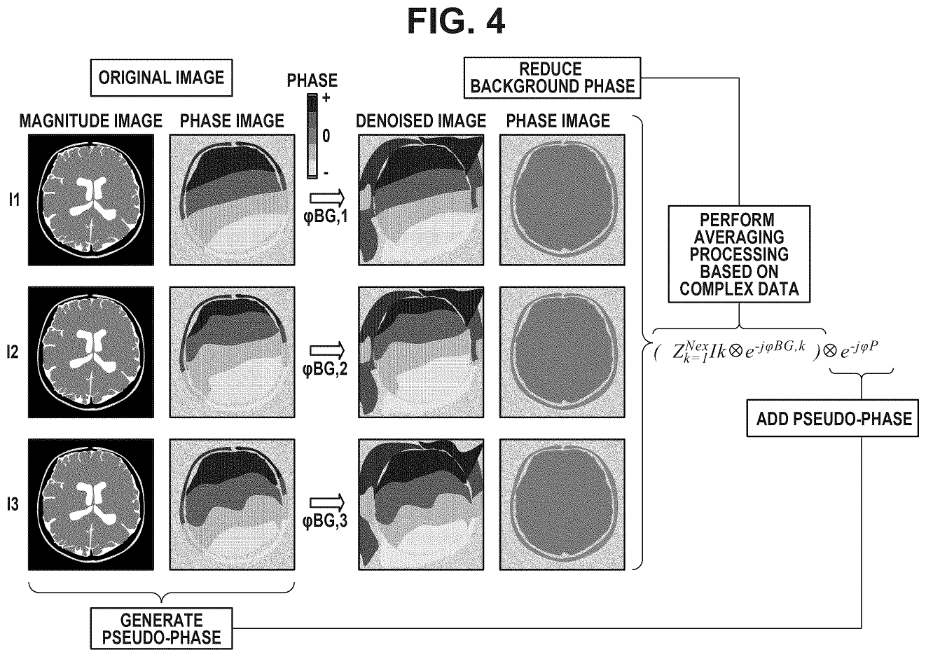

Processing corresponding to steps ST40, ST50, and ST60 illustrated in FIG. 3 will now be described with reference to FIG. 4.

The Nex is not limited to three times illustrated in FIG. 4, but instead may be any number of times greater than one. Each of first, second, and third original images I1, I2, and I3 includes a magnitude image and a phase image for each accumulation number. Equation (1) represents that an image Iavg based on one piece of complex average data is derived from the first, second, and third original images I1, I2, and I3 through processing in steps ST40, ST50, and ST60.

$$[ Equation 1 ]$$

$$Iavg = ( {\sum }_{k = I}^{N e x} Ik ⊗ {e}^{- j Φ BG , k} ) ⊗ {e}^{j ΦP} \qquad ( 1 )$$

In step ST40, the first correction function F4 performs, on at least one complex data among the multiple pieces of complex data, processing for reducing the background phase included in the complex data. The processing for reducing the background phase includes processing for removing the background phase. The first correction function F4 may perform the processing for reducing the background phase in any of data processing spaces including an image space, a k-space, and a hybrid space between the image space and the k-space. The first correction function F4 may use only the real part of the complex data.

Herein, the multiple pieces of complex data refer to complex data corresponding to the Nex. The at least one complex data among the multiple pieces of complex data refers to, for example, complex data whose accumulation number is k among complex data whose acquisition number k is 1 to Nex, and may include two or more pieces of complex data. The first correction function F4 may perform the processing for reducing the background phase on all complex data among the multiple pieces of complex data.

As illustrated in FIG. 4, the first correction function F4 estimates first, second, and third denoised images that correspond to first, second, and third original phase images, respectively, and include first, second, and third background phases ΦBG, 1, ΦBG, 2, and ΦBG, 3, respectively, and reduces the first, second, and third background phases ΦBG, 1, ΦBG, 2, and ΦBG, 3 from the first, second, and third original phase images, respectively. The first, second, and third denoised images including the first, second, and third background phases ΦBG, 1, ΦBG, 2, and ΦBG, 3, respectively, are estimated by, for example, artificial intelligence (AI), machine learning, deep learning, or interpolation processing.

The processing for reducing the background phase need not be performed on an original phase image, among the plurality of original phase images, in which the background phase does not cause image degradation in the averaging processing on the complex data in step ST50 to be described below.

In step ST50, the calculation function F5 calculates complex average data by performing averaging processing on multiple pieces of complex data including the complex data in which the background phase is reduced in step ST40. In this case, one piece of complex average data is data obtained by performing averaging processing on multiple pieces of complex data corresponding to the Nex. For example, in a case where parallel imaging processing or coil combination processing is performed not as preprocessing but as post-processing, the one piece of complex average data may be complex average data corresponding to the number of receiver coils.

As illustrated in FIG. 4, the calculation function F5 performs averaging processing on multiple pieces of complex data including first, second, and third original magnitude images and phase images obtained after the first, second, and third background phases are reduced.

In step ST60, the second correction function F6 adds a pseudo-phase to a phase corresponding to the complex average data. The pseudo-phase is a phase for maintaining the consistency between the amplitude and the phase in the complex average data. The second correction function F6 may perform processing for adding the pseudo-phase in any of data processing spaces including an image space, a k-space, and a hybrid space between the image space and the k-space. The second correction function F6 may use only real part data of the complex average data.

In Equation (1), the multiple pieces of complex data after the background phase is reduced are subjected to the averaging processing, and then the pseudo-phase is added to the complex average data. Alternatively, to the multiple pieces of complex data after the background phase is reduced, the pseudo-phase is added, and then the averaging processing may be performed thereon.

As illustrated in FIG. 4, the second correction function F6 adds the pseudo-phase based on the phase of at least one complex data among the multiple pieces of complex data to the complex average data. The pseudo-phase may be phase of at least one original image generated based on at least one complex data among the multiple pieces of complex data. Further, the pseudo-phase may be phase of an image obtained by performing at least one of denoising processing and smoothing processing on at least one original image. Specifically, the pseudo-phase is generated by, for example, the following first to fourth methods.

In the first method, as represented by Equation (2), from an original image Ik (where k=1 to Nex), a background phase ΦBG, k (where k=1 to Nex) is reduced through a denoised image, and then the averaging processing is performed thereon. Then, phase of any one of original images Ix (where x=1 to Nex) is added as a pseudo-phase. In the first method, the consistency between the amplitude and the phase may be lost, and there is a risk that an artifact such as a severe black dropout of a signal may occur in the MR image.

$$[ Equation 2 ]$$

$$Iavg = ( {\sum }_{k = 1}^{N e x} Ik ⊗ {e}^{- j Φ BG , k} ) ⊗ {e}^{j arg ( Ix )} \qquad ( 2 )$$

In the second method, as represented by Equation (3), from the original image Ik (where k=1 to Nex), a background phase ΦBG, k (where k=1 to Nex) is reduced through the denoised image, and then the averaging processing is performed thereon. Then, any one of background phases ΦBG, x (where x=1 to Nex) through a denoised image is added as a pseudo-phase. In the second method, severe black dropout of a signal can be reduced compared to the first method although there is a risk that a smoothed MR image with a blur will be generated due to addition of the phase of the denoised image.

$$[ Equation 3 ]$$

$$Iavg = ( {\sum }_{k = 1}^{N e x} Ik ⊗ {e}^{- j Φ BG , k} ) ⊗ {e}^{j Φ BG , x} \qquad ( 3 )$$

In the third method, as represented by Equation (4), from the original image Ik (where k=1 to Nex), the background phase ΦBG, k (where k=1 to Nex) is reduced through the denoised image, and then averaging processing is performed thereon. Then, a phase obtained by averaging denoised images Dk (i.e., background phases ΦBG, k) (where k=1 to Nex) is added as the pseudo-phase. In the second method, the phase of one denoised image is added as the pseudo-phase, whereas in the third method, the phase obtained by averaging the denoised images is added as the pseudo-phase, which makes it possible to add a more stable pseudo-phase.

$$[ Equation 4 ]$$

$$Iavg = ( {\sum }_{k = 1}^{N e x} Ik ⊗ {e}^{- j Φ BG , k} ) ⊗ {e}^{j arg ( \frac{1}{N e x} {\sum }_{k = 1}^{N e x} D ( 1 = Ik ) )} \qquad ( 4 )$$

In the fourth method, as represented by Equation (5), from the original image Ik (where k=1 to Nex), the background phase ΦBG, k (where k=1 to Nex) is reduced through the denoised image, and further, smoothing processing for weighting with a coefficient Wk according to a signal amount represented by Equation (6) is performed. Then, the averaging processing is performed thereon. Then, the phase obtained by averaging the denoised images Dk (i.e., background phases ΦBG, k) (where k=1 to Nex) is added as the pseudo-phase. In the fourth method, occurrence of a flow omission due to a pulsation, blood flow, or the like can be suppressed compared to the third method.

$$[ Equation 5 ]$$

$$Iavg = ( {\sum }_{k = 1}^{N e x} Ik ⊗ {e}^{- j Φ BG , k} ) ⊗ {e}^{j arg ( \frac{1}{N e x} {\sum }_{k = 1}^{N e x} D ( Ik ) ⊗ Wk )} \qquad ( 5 )$$

$$\frac{{\vert S ( Ik ) \vert}^{2}}{{\sum }_{k = 1}^{N e x} {\vert S ( Ik ) \vert}^{2}} \qquad ( 6 )$$

As described above, the setting of whether to execute the processing for reducing the background phase in step ST20 may be performed after the processing for reducing the background phase. For example, if the artifact is not reduced or is increased as a result of executing the processing for reducing the background phase, the setting function F2 may perform a setting not to execute the processing for reducing the background phase.

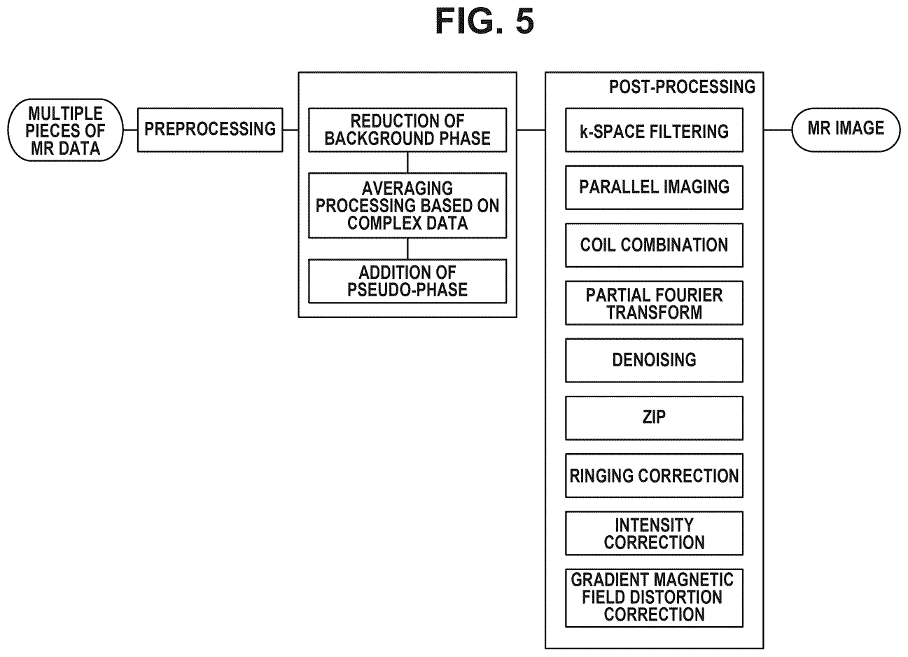

The description continues with reference again to FIG. 3. In step ST70, the post-processing function F7 performs post-processing on the complex average data. As illustrated in FIG. 5, the post-processing is at least one type of processing among k-space filtering processing, parallel imaging processing, coil combination processing, partial Fourier transform processing, denoising processing, ZIP processing, ringing correction processing, signal intensity correction processing, and gradient magnetic field distortion correction processing.

In step ST70, in a case where post-processing that requires phase information is not to be performed, addition of the pseudo-phase in step ST60 may be omitted. The post-processing function F7 may perform processing for reducing the pseudo-phase added to the complex average data. The processing for reducing the pseudo-phase includes processing for removing the pseudo-phase. The post-processing function F7 may use only the real part data of the complex average data.

The k-space filtering processing is filter processing on MR data corresponding to a specific frequency component or a phase component of a characteristic on k-space, such as high-frequency filtering, low-frequency filtering, or band-pass filtering.

In the parallel imaging processing, MR data is collected while being thinned out by a plurality of receiver coils to reduce scan time. In parallel imaging processing, a lack of MR data in k-space is compensated for by an algorithm such as SENSitivity Encoding (SENSE) or GeneRalized Autocalibrating Partially Parallel Acquisitions (GRAPPA). In the coil combination processing, multiple pieces of MR data collected by a plurality of receiver coils are appropriately weighted based on coil sensitivity, and are combined. The coil combination processing may be performed as a part of the parallel imaging processing.

In the partial Fourier transform processing, some pieces of MR data in k-space are collected to compensate for other pieces of the MR data in k-space that are not collected yet. In the denoising processing, for example, a noise component included in an MR image is estimated by deep learning, and the noise component is selectively reduced. In the ZIP processing, the size of data is increased to be larger than k-space of the MR data to be collected to fill the outside of the k-space of the MR data to be collected with “0”. In the ringing correction processing, image blurring, ringing artifacts, and the like that appear after the ZIP processing are reduced by, for example, a convolutional neural network (CNN) that is trained by supervised learning to be specialized for ZIP processing.

In the signal intensity correction processing, fluctuations in signal intensity caused by unevenness of coil sensitivity, a difference depending on a region in the body of a patient, unevenness of a static magnetic field, an imaging condition, and the like are corrected. In the gradient magnetic field distortion correction processing, a distortion caused in an MR image due to unevenness of a gradient magnetic field, non-linearity, or the like is corrected.

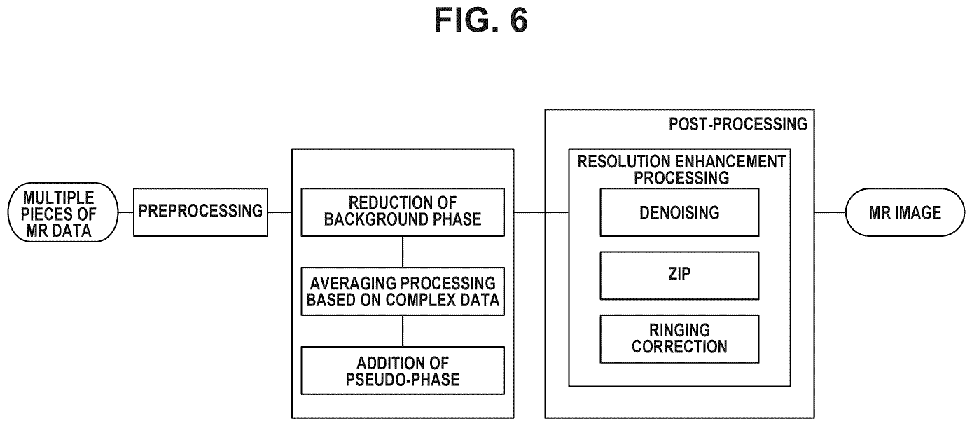

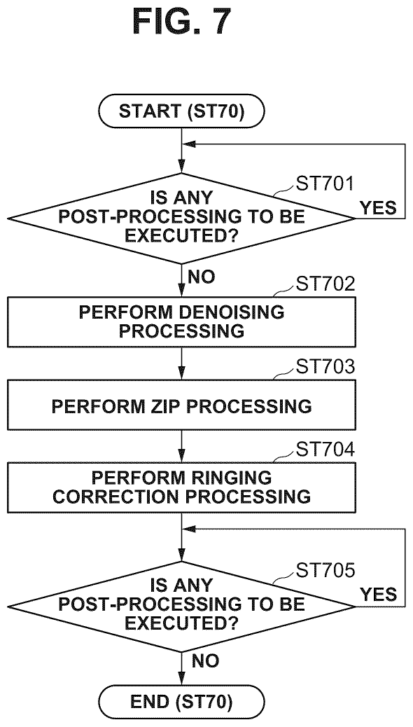

As illustrated in FIG. 6, the post-processing function F7 can perform the resolution enhancement processing using the denoising processing, the ZIP processing, and the ringing correction processing on the complex average data. The resolution enhancement processing is performed by, for example, AI, machine learning, deep learning, or interpolation processing. An example of operation in step ST70 illustrated in FIG. 3 in which the resolution enhancement processing is executed in the post-processing will be described with reference to the flowchart illustrated in FIG. 7.

In step ST701, the post-processing function F7 determines whether to execute any type of post-processing other than the resolution enhancement processing among the types of post-processing is to be executed. In step ST701, if all the types of post-processing to be executed prior to step ST702 have been completed (NO in step ST701), the processing proceeds to step ST702.

In step ST702, the post-processing function F7 performs the denoising processing. In step ST703, the post-processing function F7 performs the ZIP processing. In step ST704, the post-processing function F7 performs the ringing correction processing. The resolution enhancement processing in steps ST702 to ST704 can be performed at an early stage after complex average data is calculated by the averaging processing. In step ST701, the resolution enhancement processing may be performed immediately after step ST60 without executing any type of the post-processing.

In step ST705, the post-processing function F7 determines whether any type of post-processing other than the resolution enhancement processing among the types of post-processing is to be executed. In step ST705, if all types of the post-processing have been completed (NO in step ST705), the processing of step ST70 is completed.

As described above, the setting of whether to execute the resolution enhancement processing in step ST20 may be performed after the processing for reducing the background phase. For example, if the amount of artifacts generated is increased as a result of performing the resolution enhancement processing, the setting function F2 may perform a setting not to perform the resolution enhancement processing. Alternatively, the probability of occurrence of the artifacts may be numerically expressed, and the setting function F2 may perform the setting to execute the resolution enhancement processing when the probability of occurrence of the artifacts is higher than or equal to a threshold.

The description continues with reference again to FIG. 3. In step ST80, the display control function F8 performs control to display a reconstructed MR image on the display 45.

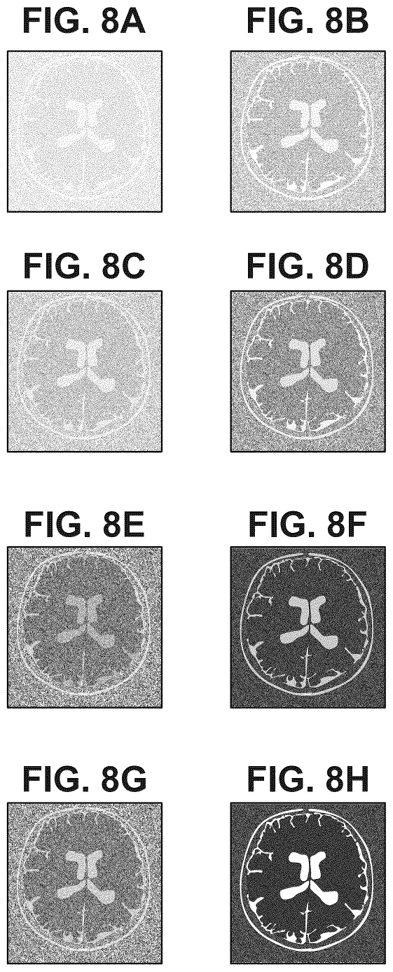

FIGS. 8A to 8H each illustrate an example of a change in image quality of an MR image according to the type of averaging processing and the Nex. FIGS. 8A, 8C, 8E, and 8G schematically illustrate a case where the Nex is “2”, and FIGS. 8B, 8D, 8F, and 8H each schematically illustrate a case where the Nex is “10”.

FIGS. 8A and 8B each illustrate a case where the averaging processing is executed based on absolute value data. In the case of executing the averaging processing based on the absolute value data, a noise distribution does not follow a Gaussian distribution. Thus, when the Nex is increased, the noise texture is reduced, but a whitish area tends to remain in the entire MR image.

FIGS. 8C and 8D each illustrate a case where the averaging processing is executed based on complex data. In the case of executing the averaging processing based on the complex data, background noise can be reduced by increasing the Nex, so that there is a tendency that a whitish area does not remain in the entire MR image compared with the case where the averaging processing is executed based on the absolute value data.

FIGS. 8E and 8F each illustrate a case where the averaging processing based on the complex data is executed after the denoising processing. FIGS. 8G and 8H each illustrate a case where the denoising processing is performed after the averaging processing based on the complex data. In the case of executing the denoising processing after the averaging processing based on the complex data, the whitish area in the entire MR image is reduced, and image blurring, which is caused when the averaging processing based on the complex data is executed after the denoising processing, is reduced, so that a sharper MR image can be generated.

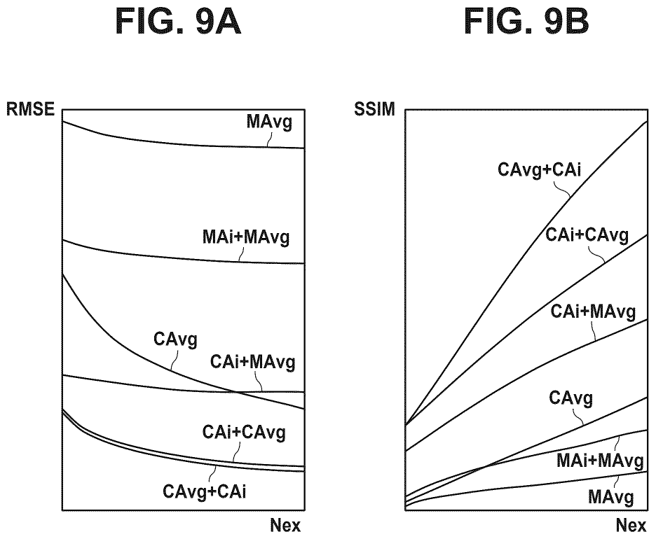

FIGS. 9A and 9B are graphs each illustrating an example of a change in image quality of an MR image according to the type of averaging processing and the Nex. The image quality of each of an MR image generated when the averaging processing based on the absolute value data is performed is represented by “MAvg”, an MR image generated when the averaging processing based on the absolute value data is performed after the resolution enhancement processing based on the absolute value data is represented by “MAi+MAvg”, an MR image generated when the averaging processing based on the complex data is performed is represented by “CAvg”, an MR image generated when the averaging processing based on the absolute value data is performed after the resolution enhancement processing based on the complex data is represented by “CAi+MAvg”, an MR image generated when the averaging processing based on the complex data is performed after the resolution enhancement processing based on the complex data is represented by “CAi+CAvg”, and an MR image generated when the resolution enhancement processing based on the complex data is performed after the averaging processing based on the complex data is represented by “CAi+CAvg”. The generated MR images are evaluated by Root Mean Square Error (RMSE) in FIG. 9A and are evaluated by Structural Similarity Index Measure (SSIM) in FIG. 9B.

FIG. 9A illustrates that the RMSE tends to decrease and the image quality tends to improve as the Nex is increased for all the types of averaging processing. Further, in the MR image generated when the resolution enhancement processing based on the complex data is performed after the averaging processing based on the complex data, which is represented by “CAi+CAvg”, the RMSE tends to further decrease, and the image quality tends to improve. FIG. 9B illustrates that the SSIM tends to increase and the image quality tends to improve as the Nex is increased for all the types of averaging processing. Further, in the MR image generated when the resolution enhancement processing based on the complex data is performed after the averaging processing based on the complex data, which is represented by “CAi+CAvg”, the SSIM tends to further increase, and the image quality tends to improve.

Since the image processing apparatus 4 according to the embodiment executes the averaging processing prior to the post-processing, it is expected that image quality of an MR image to be generated is improved as a result of improvement in accuracy of image processing in the subsequent post-processing, and that the processing speed is improved by 1/Nex. Further, the image processing apparatus 4 according to the embodiment adds a pseudo-phase after the averaging processing, thereby making it possible to generate a sharper MR image with higher image quality than conventional MR images even in a case where post-processing that requires phase information, such as ZIP processing, is performed.

### (Image Processing Apparatus According to Modified Example of Embodiment)

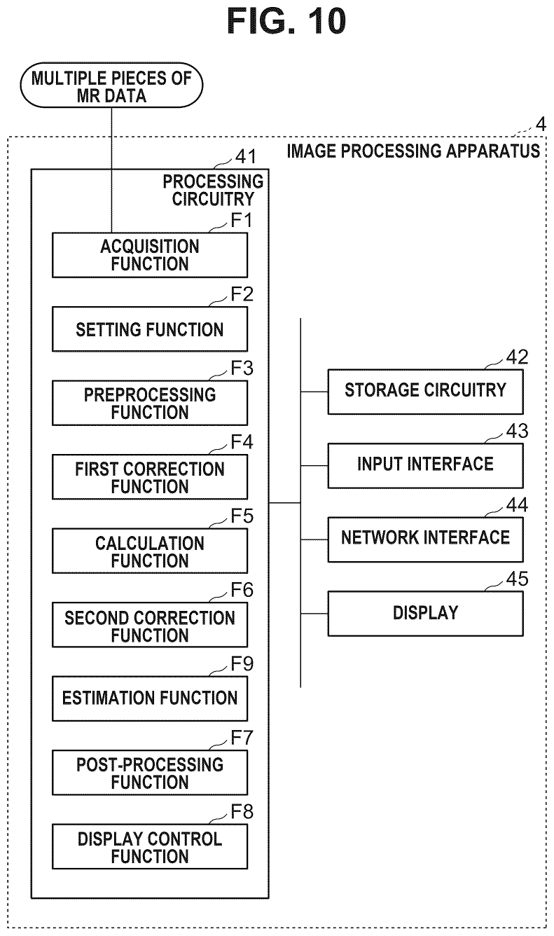

In the image processing apparatus 4 according to the embodiment, even when the pseudo-phase generated by the first to fourth methods is added, an artifact can remain in some areas of the MR image to be generated. Thus, in a modified example of the embodiment in which a pseudo-phase is generated by a fifth method, the pseudo-phase is locally smoothed to such an extent that an artifact or a missing signal does not occur in the MR image to be generated. FIG. 10 is a block diagram illustrating an example of an overall configuration of the image processing apparatus 4 according to the modified example of the embodiment. In the modified example of the embodiment, the processing circuitry 41 further executes an estimation function F9.

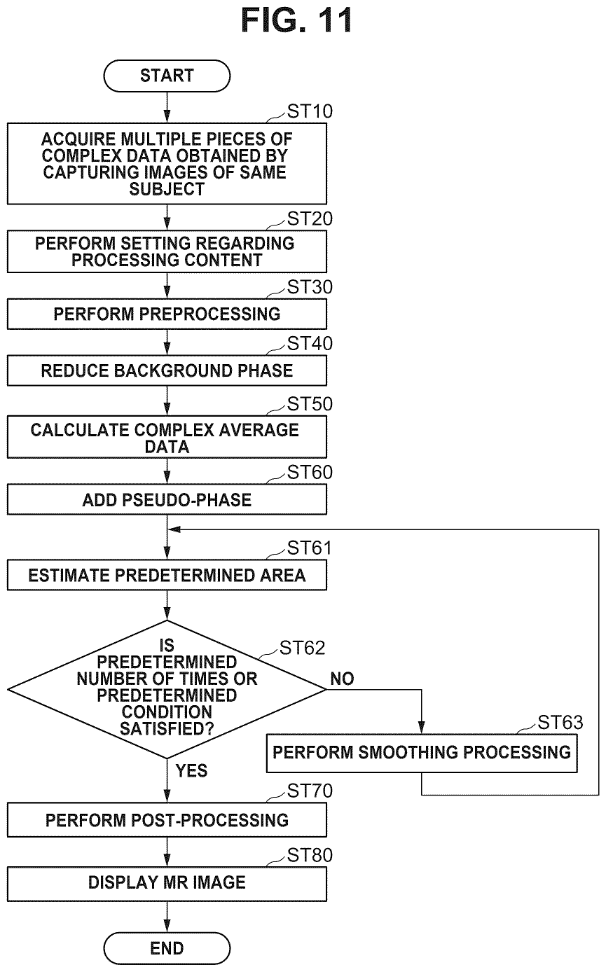

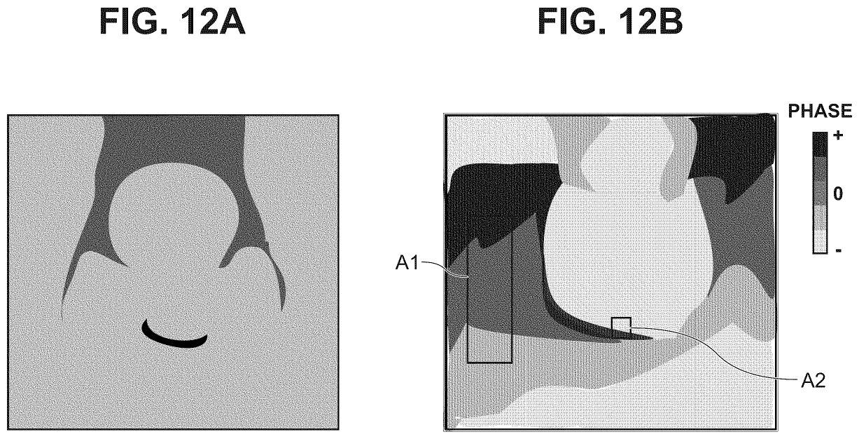

An example of operation with regard to generation of an MR image based on multiple pieces of MR data will be described with reference to the flowchart illustrated in FIG. 11 and FIGS. 12A and 12B. In the modified example of the embodiment, the processing proceeds to step ST61 after step ST60.

In step ST61, the estimation function F9 estimates, as a predetermined area, an area where an artifact or a missing signal due to a discontinuous phase change occurs in an image based on the complex average data or an image based on data obtained after a pseudo-phase is added. The predetermined area may be estimated by comparing between an MR image based on the complex average data and an MR image based on the data obtained after a pseudo-phase is added, or may be estimated by a phase pole in a phase image.

The phase pole is a point where there is no smooth continuous phase change, but a discontinuous phase change such that the phase abruptly changes between a specific voxel or pixel and surrounding voxels or pixels. FIG. 12A illustrates an example of a magnitude image of a specific tissue, and FIG. 12B illustrates an example of a phase image corresponding to FIG. 12A. As illustrated in FIG. 12B, for example, in an area where there is a signal due to a tissue, the phase smoothly changes (e.g., area A1), whereas in an area where there is no signal due to a tissue, the phase rapidly changes (e.g., area A2). A boundary of the area A2 is an example of the phase pole.

In step ST62, the estimation function F9 determines whether a predetermined number of times or a predetermined condition is satisfied. The predetermined condition is a condition under which an index (e.g., a pixel value) obtained by quantifying the number and a probability of occurrence of areas each estimated as an area where an artifact or a missing signal due to a discontinuous phase change occurs is less than or equal to a threshold.

In step ST63, the second correction function F6 further performs smoothing processing on the predetermined area of the image based on the complex average data or the image based on the data obtained after the pseudo-phase is added. More specifically, the second correction function F6 repeatedly performs the smoothing processing on the predetermined area until the smoothing processing is executed the predetermined number of times or the predetermined condition is satisfied. In the modified example of the embodiment, the processing proceeds to step ST70 after step ST63.

The image processing apparatus 4 according to the modified example of the embodiment locally performs smoothing processing on the pseudo-phase, thereby making it possible to further reduce degradation in image quality of an MR image, such as an artifact and a missing signal, compared to the image processing apparatus 4 according to the embodiment.

### (MRI Apparatus)

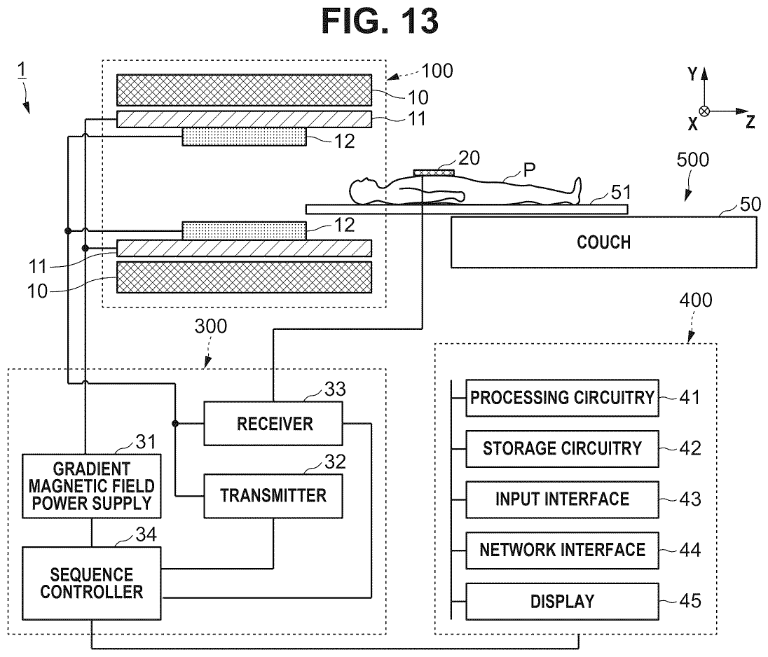

In a magnetic resonance imaging (MRI) apparatus 1 according to another embodiment, a console 400 includes the image processing apparatus 4. The MRI apparatus 1 collects multiple pieces of complex data obtained by the image processing apparatus 4 capturing images of the same subject. FIG. 13 is a block diagram illustrating an example of an overall configuration of the MRI apparatus 1 according to the embodiment. The MRI apparatus 1 includes a gantry apparatus 100, a control cabinet 300, the console 400, and a couch 500. Specifically, the console 400 includes the processing circuitry 41, the storage circuitry 42, the input interface 43, the network interface 44, and the display 45. The MRI apparatus 1 may be configured to be capable of communicating with the image processing apparatus 4 installed in a remote location via a network.

The gantry apparatus 100 includes a static field magnet 10, a gradient coil 11, and a whole body (WB) coil 12. These constituent elements are housed in a cylindrical housing.

The static field magnet 10 has a substantially cylindrical shape and generates a static magnetic field in a bore into which a patient to be a subject P is carried. The bore is an examination space in the cylindrical shape of the static field magnet 10. The static field magnet 10 incorporates a superconductive coil. The superconductive coil is cooled to very low temperature by liquid helium. The static field magnet 10 generates a static magnetic field by applying an electric current supplied from a static magnetic field power supply (not illustrated) to the superconductive coil in an excitation mode. After that, when the static field magnet 10 transitions to a permanent current mode, the static magnetic field power supply is disconnected. Once the static field magnet 10 transitions to the permanent current mode, the static field magnet 10 continues to generate a large static magnetic field for a long period of time, for example, for one year or more. The static field magnet 10 may be composed of a permanent magnet.

The gradient coil 11 has a substantially cylindrical shape and is fixed to an inner side of the static field magnet 10 in a radial direction of the cylindrical shape. The gradient coil 11 receives an electric current supplied from a gradient magnetic field power supply 31 and generates a gradient magnetic field. The gradient coil 11 is formed by combining three coils respectively corresponding to X-, Y-, and Z-axes that are orthogonal to each other. The three coils individually receive respective electric currents supplied from the gradient magnetic field power supply 31 and generate gradient magnetic fields whose magnetic field strengths vary along the X-, Y-, and Z-axes.

In this case, the Z-axis direction is a direction set along a magnetic flux of a static magnetic field generated by the static field magnet 10, and is the same direction as a longitudinal direction of a couchtop 51. The Y-axis direction is a vertical direction orthogonal to the Z-axis direction and is a direction perpendicular to the couchtop 51. The X-axis direction is a direction orthogonal to both the Z-axis and Y-axis directions and is the same direction as the lateral direction of the couchtop 51.

The WB coil 12 is a radio frequency (RF) coil that has a substantially cylindrical shape and is fixed so as to surround the subject P on the inner side of the gradient coil 11. The WB coil 12 transmits RF pulses transmitted from a transmitter 32 to the subject P and receives MR signals emitted from the subject P by excitation of hydrogen nuclei.

The MRI apparatus 1 may include a local coil 20 in addition to the WB coil 12. The local coil 20 is an RF coil disposed in proximity to the subject P and receives MR signals emitted from the subject P at a position close to the subject P. The local coil 20 may transmit RF pulses transmitted from the transmitter 32 to the subject P. As the local coil 20, various types of local coils can be used according to an imaging region of the subject P. Examples of the local coils include a local coil for head, a local coil for chest (e.g., see FIG. 13), a local coil for spine, a local coil for lower extremities, and a local coil for whole body.

The control cabinet 300 includes the gradient magnetic field power supply 31, the transmitter 32, a receiver 33, and a sequence controller 34. The gradient magnetic field power supply 31 supplies an electric current to the gradient coil 11 under the control of the sequence controller 34, and generates a gradient magnetic field along each of the X-, Y-, and Z-axes.

The transmitter 32 generates an RF pulse train in the Larmor frequency band as an RF transmission wave based on an instruction from the sequence controller 34, and outputs RF pulses to the RF coil to thereby excite the subject P.

The receiver 33 performs an analog-to-digital (AD) conversion on the MR signals received by the RF coil and outputs digitized signals to the sequence controller 34. The digitized MR signal is referred to as raw data.

The sequence controller 34 drives each of the gradient magnetic field power supply 31, the transmitter 32, and the receiver 33 under the control of the console 400, thereby executing scanning of the subject P. The sequence controller 34 receives the raw data via the receiver 33 and transmits the raw data to the console 400.

The sequence controller 34 includes processing circuitry (not illustrated). The processing circuitry of the sequence controller 34 is configured with, for example, hardware such as a processor that executes a predetermined program, a field programmable gate array (FPGA), or an application specific integrated circuit (ASIC).

The couch 500 includes a couch main body 50 and the couchtop 51. The couch main body 50 is configured to move the couchtop 51 in the vertical direction and the horizontal direction, and moves the subject P placed on the couchtop 51 to a predetermined height to thereby move the subject P into the bore.

These constituent elements enable the console 400 to control the entire MRI apparatus 1. The processing circuitry 41 of the console 400 includes the functions of the processing circuitry 41 of the image processing apparatus 4, and further includes a function for controlling the entire MRI apparatus 1. The processing circuitry 41 of the console 400 receives an instruction with regard to imaging conditions through an operation by a user such as a radiologic technologist, via the input interface 43. Then, the processing circuitry 41 causes the sequence controller 34 to execute scanning based on the input imaging conditions, and collects multiple pieces of complex data on the same subject. Further, the processing circuitry 41 generates an MR image based on raw data transmitted from the sequence controller 34. The generated MR image is displayed on the display 45 and is stored in the storage circuitry 42.

The image processing apparatus and the phase correction method according to at least one of the embodiments described above can improve the image quality without extending a processing time until an MR image is generated.

In the above embodiments, the term “processor” means, for example, circuitry such as a dedicated or general-purpose central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)).

When the processor is, for example, a CPU, the processor reads and executes a program stored in storage circuitry to implement various functions. When the processor is, for example, an ASIC, a function corresponding to the program is directly incorporated as logic circuitry in circuitry of the processor instead of the processor storing the program in the storage circuit. In this case, the processor implements various functions by hardware processing of reading and executing the program incorporated in the circuitry, or the processor can also implement various functions by combining software processing and hardware processing.

In the embodiments described above, the example is described in which the single processor of the processing circuitry implements the functions. However, the processing circuitry may be configured by combining a plurality of independent processors, and the processors may implement the respective functions. In a case where the plurality of processors is provided, the storage circuitry that stores the programs may be provided individually for each processor, or one piece of storage circuitry may collectively store the programs corresponding to the functions of all the processors.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing apparatus comprising processing circuitry configured to:
perform processing for reducing a background phase included in complex data, the complex data being at least one piece of complex data among multiple pieces of complex data obtained by scanning a subject by a magnetic resonance imaging apparatus;
calculate complex average data by performing averaging processing on the multiple pieces of complex data including the complex data in which the background phase is reduced; and
perform post-processing on the complex average data.

2. The image processing apparatus according to claim 1, wherein the processing circuitry is further configured to add a pseudo-phase to a phase corresponding to the complex average data.

3. The image processing apparatus according to claim 2, wherein the processing circuitry is configured to add the pseudo-phase to the phase corresponding to the complex average data prior to performing the post-processing and after calculating the complex average data.

4. The image processing apparatus according to claim 2, wherein the pseudo-phase is a phase based on a phase corresponding to the at least one piece of complex data among the multiple pieces of complex data.

5. The image processing apparatus according to claim 2, wherein the pseudo-phase is a phase corresponding to an image generated based on the at least one piece of complex data among the multiple pieces of complex data.

6. The image processing apparatus according to claim 2, wherein the pseudo-phase is a phase corresponding to an image obtained by performing at least one of denoising processing and smoothing processing on an image generated based on the at least one piece of complex data among the multiple pieces of complex data.

7. The image processing apparatus according to claim 1, wherein the post-processing is at least one of k-space filtering processing, parallel imaging processing, coil combination processing, partial Fourier transform processing, denoising processing, Zero Filling Interpolation (ZIP) processing, ringing correction processing, signal intensity correction processing, and gradient magnetic field distortion correction processing.

8. The image processing apparatus according to claim 1, wherein the processing circuitry is further configured to perform the processing for reducing the background phase in one of data processing spaces including an image space, a k-space, and a hybrid space between the image space and the k-space.

9. The image processing apparatus according to claim 2, wherein the processing circuitry is further configured to perform processing for adding the pseudo-phase in one of data processing spaces including an image space, a k-space, and a hybrid space between the image space and the k-space.

10. The image processing apparatus according to claim 1, wherein the processing circuitry is further configured to perform resolution enhancement processing using denoising processing, ZIP processing, and ringing correction processing on the complex average data.

11. The image processing apparatus according to claim 1, wherein the processing circuitry is further configured to perform the processing for reducing the background phase on all of the multiple pieces of complex data.

12. The image processing apparatus according to claim 2, wherein the processing circuitry performs processing for adding the pseudo-phase on all of the multiple pieces of complex data.

13. The image processing apparatus according to claim 1, wherein the processing circuitry is further configured to perform smoothing processing on a predetermined area of an image based on the complex average data, the predetermined area being an area where an artifact or a missing signal due to a discontinuous phase change occurs in the image based on the complex average data.

14. The image processing apparatus according to claim 13, wherein the processing circuitry is further configured to repeatedly perform the smoothing processing on the predetermined area until the smoothing processing is executed a predetermined number of times or a predetermined condition is satisfied, the predetermined condition being a condition in which an index obtained by quantifying a number and an occurrence probability of areas estimated to be regions where an artifact or a missing signal due to a discontinuous phase change occurs is less than or equal to a threshold.

15. The image processing apparatus according to claim 2, wherein the processing circuitry is further configured to perform processing for reducing the pseudo-phase added to the complex average data.

16. A phase correction method comprising:
performing processing for reducing a background phase included in complex data, the complex data being at least one piece of complex data among multiple pieces of complex data obtained by scanning a subject by a magnetic resonance imaging apparatus;
calculating complex average data by performing averaging processing on the multiple pieces of complex data including the complex data in which the background phase is reduced; and
performing post-processing on the complex average data.
